(19) United States
(12) Patent Application Publication
(10) Pub. No.: US 2021/0198692 A1
(43) Pub. Date: Jul. 1, 2021

(54) ADENO-ASSOCIATED VIRAL VECTOR-MEDIATED GENE THERAPY FOR TREATING FRAGILE X-ASSOCIATED DISORDERS

(71) Applicant: THE GOVERNING COUNCIL OF THE UNIVERSITY OF TORONTO, Toronto (CA)

(72) Inventors: David R. HAMPSON, Toronto (CA); Shervin GHOLIZADEH MOGHADDAM, Toronto (CA); Jason ARSENAULT, Toronto (CA); Yosuke NIIBORI, Toronto (CA); Alexander W.M. HOOPER, Toronto (CA)

(21) Appl. No.: 17/057,346

(22) PCT Filed: May 30, 2019
(86) PCT No.: PCT/CA2019/050741

§ 371 Date: Nov. 20, 2020

(30) Foreign Application Priority Data
May 31, 2018 (GB) 1808943.3

(51) Int. Cl. C12N 15/86; C12N 7/00; C07K 14/47; A61K 9/00; A61P 25/00

(52) U.S. Cl. CPC C12N 15/86; C12N 7/00; C07K 14/47; C12N 2750/14123; A61P 25/00; C12N 2750/14143; C12N 2750/14171; A61K 9/0085

(57) ABSTRACT

The present application provides adeno-associated viral vector-mediated gene therapy for treating Fragile X-associated disorders, including Fragile X Syndrome (FXS). In particular, there is provided a vector comprising an adeno-associated vims (AAV) genome or a derivative thereof and a nucleic acid sequence encoding a Fragile X Mental Retardation Protein (FMRP) isoform that lacks exon 12 and includes exon 14 of the full length FMRP gene, such as a human or a murine Group C FMRP isoform. Also provided are related pharmaceutical compositions comprising this vector, and methods and uses thereof for the treatment of Fragile X-associated disorders, including FXS.

### CROSS-REFERENCE TO RELATED APPLICATIONS

This application is a 35 U.S.C. § 371 filing of International Patent Application No. PCT/CA2019/050741, filed May 30, 2019, which claims priority from United Kingdom Patent Application No. 1808943.3, filed May 31, 2018, the entire disclosures of which are hereby incorporated herein by reference.

### FIELD OF THE INVENTION

The present application pertains to the field of gene therapy. More particularly, the present application relates to gene therapy in the treatment of Fragile X-associated disorders, including Fragile X Syndrome, and vectors and compositions for use therein.

### INTRODUCTION

Fragile X-associated disorders are caused by mutations in the fragile X mental retardation 1 (FMR1) gene. This gene encodes a protein called the Fragile X Mental Retardation Protein (FMRP), which is required for normal brain development. Fragile X-associated disorders include Fragile X Syndrome (FXS), Fragile X-associated primary ovarian insufficiency (FXPOI) and Fragile X-associated tremor/ataxia syndrome (FXTAS).

Although considered a “rare disorder”, FXS is the most frequent single-gene cause of cognitive impairment and autism, affecting about 1 in 6,000 newborns. FXS is a neurodevelopmental disorder caused by a full mutation in the FMR1 gene, in which a genomic DNA trinucleotide repeat (CGG) expansion to 200 or more CGG repeats in the FMR1 gene causes the abrogation of FMRP expression in the body, including the brain where it is highly expressed. Thus, FMRP is missing or drastically reduced in FXS and its absence or reduction in the brain is the cause of the most serious symptoms: cognitive impairment, autistic behaviors including stereotypy and communication and language impairments, seizures, and other features. These deficits and medical abnormalities extend throughout the lifespan of affected individuals resulting in the need for life-long care. Currently, there is no treatment that effectively addresses the cognitive impairment and autistic behaviors of individuals with FXS.

Other Fragile X-associated disorders are associated with changes in the FMR1 gene, such that it is not turned off but it does not function normally. These disorders can be caused by a premutation of the FMR1 gene, for example, in which the gene has greater than about 60 CCG repeats, but less than 200 CCG repeats. A range of clinical conditions can result from premutations in the FMR1 gene, including FXPOI, FXTAS, psychiatric problems, hypertension, migraines and autoimmune problems.

FXPOI is a cause of infertility and early menopause in young women. Also, women who have a premutation in the FMR1 gene are more likely to have children who have FXS. FXTAS is a disorder of the nervous system that can cause tremors and problems with balance (or ataxia), memory, and mood disorders. Again, FXTAS can be caused by a premutation in the FMR1 gene.

Typically, the treatment of Fragile X-associated disorders is directed at the symptoms only. Treatments that target the cause of Fragile X-associated disorders are now being studied, often using Fmrl knockout (KO) mice. However, to date, an effective treatment has not yet been identified.

The above information is provided for the purpose of making known information believed by the applicant to be of possible relevance to the present invention. No admission is necessarily intended, nor should be construed, that any of the preceding information constitutes prior art against the present invention.

### SUMMARY

An object of the present application is to provide adeno-associated viral vector-mediated gene therapy for treating Fragile X-associated disorders, including Fragile X Syndrome (FXS). In accordance with an aspect of the present application, there is provided a vector comprising an adeno-associated virus (AAV) genome or a derivative thereof and a nucleic acid sequence encoding a Fragile X Mental Retardation Protein (FMRP) isoform that lacks exon 12 and includes exon 14 of the full length FMRP1 gene. The vector is referred to herein as an AAV-FMRP vector. Optionally the FMRP isoform is a human or a murine Group C FMRP isoform, such as human FMRP isoform 17 or murine FMRP isorform 7 (Pretto, D. I., et al. (2015) J Med Genet 52(1): 42-52). The AAV-FMRP vector is particularly suitable for use in treating or preventing a Fragile X-associated disorder, such as FXS, in a subject in need thereof.

In accordance with another aspect, there is provided a method of treating or preventing a Fragile X-associated disorder (such as FXS) in a subject in need thereof, comprising administering a therapeutically effective amount of an AAV-FMRP vector as described herein to the subject. Optionally, the method comprises administering the AAV-FMRP vector directly into the CNS of the subject, for example, by intra-cerebroventricular injection (ICV), intra-cisterna magna injection (ICM), and/or intrathecal injection.

In a related aspect, there is provided a use of an AAV-FMRP vector as described herein for treating or preventing a Fragile X-associated disorder in a subject in need thereof

In accordance with another aspect, there is provided a pharmaceutical composition comprising: (i) an AAV-FMRP vector, wherein the AAV-FMRP vector comprising an adeno-associated virus (AAV) genome or a derivative thereof and a nucleic acid sequence encoding a Fragile X Mental Retardation Protein (FMRP) isoform that lacks exon 12 and includes exon 14 of the full length FMRP1 gene; and (ii) a pharmaceutically acceptable carrier or excipient. Optionally, the pharmaceutical composition is formulated for direct administration to the CNS of a subject, such as by intra-cerebroventricular injection, intra-cisterna magna injection, and/or intrathecal injection.

### BRIEF DESCRIPTION OF FIGURES

For a better understanding of the application as described herein, as well as other aspects and further features thereof, reference is made to the following description which is to be used in conjunction with the accompanying drawings, where:

FIG. 1 is a schematic diagram of the FMR1 gene (D. Pretto et al 2015) showing protein coding exons and various FMRP isoforms produced via alternative splicing;

FIG. 2A depicts the polynucleotide sequence encoding FMRP mouse isorform 7; and FIG. 2B depicts the polynucleotide sequence encoding FMRP human isoform 17;

FIG. 3A is a schematic diagram of AAV-FMRP vector miniMeCP2-FMRP Iso7-AAV9; FIG. 3B depicts the DNA sequence of AAV-FMRP vector miniMeCP2-FMRP Iso7-AAV9, and FIG. 3C depicts the protein sequence of the protein transcribed from AAV-FMRP vector miniMeCP2-FMRP Iso7-AAV9;

FIGS. 4A-4B depict hippocampal expression levels of total FMRP in wild-type mice and Fmrl-KO mice treated with AAV-FMRPiso7, where FIG. 4A depicts a western blot of brain hippocampal lystates from the mice and FIG. 4B graphically shows the results from quantification of total FMRP from the protein bands in the western blot shown in 4A;

FIG. 5 is a photomicrograph of sagittal brain section from neonatal Fmrl-KO mouse pup treated with AAV-FMRPiso7, where the brain section was stained with anti-FMRP antibody and visualized using immunofluorescence (20× objective magnification);

FIGS. 6A-6F are photomicrographs that depict natural cellular co-distribution of MeCP2 and FMRP protein expression in wild-type mouse brain, where the photomicrographs are of thin sections from the cerebral cortex (FIG. 6A), striatum (FIG. 6B), and hippocampus (FIG. 6C), and of the deep cerebellar nuclei (DCN) under low (FIG. 6D) and high magnification (FIG. 6E), and of the cerebellar cortex showing the Purkinje cell layer situated between the molecular layer (on the right side in each panel of FIG. 6F) and granule cell layer (left side of each panel in FIG. 6F);

FIGS. 7A-7D depict immunocytochemical analysis of a Fmrl-KO mouse treated with AAV-FMRPiso 7 depicting expression in thin sections of brain using anti-FMRP with anti-cell type marker antibodies and double-label immunofluorescence, where FIGS. 7A-7C are representative images of cerebral cortex labelled for NeuN (all neurons), GAD65/67 (GABAergic neurons only), and S100β (glia), respectively, and co-labelled for FMRP (5C2 anti-FMRP antibody) (arrows indicate examples of cells positively stained for GAD65/67; arrowhead indicates example of GAD65/67 positive cell co-labelled for FMRP; scale bar=50 μm and FIG. 7D graphically represents quantification of FMRP and cell type marker co-localization by brain region (RSP/VIS=retrosplenial/visual cortex, SS/MO=somatosensory/motor cortex, CA1=hippocampus CA1 region, CA3=hippocampus CA3 region; Error bars=SEM, n=4 for all groups, * P<0.05 (ANOVA and Tukey's HSD); and

FIG. 8 depicts a western blot of liver lystates from wild-type mice and Fmrl-KO mice treated with AAV-FMRP, which demonstrated that AAV-FMRP is not expressed in the liver subsequent to AAV-FMRP injection into the cerebral spinal fluid. Fmrl-KO-EV, Fmrl KO mouse injected with negative control “empty vector” (EV).

### DETAILED DESCRIPTION

Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “Fragile X-associated disorder” refers to any disorder associated with changes in the FMR1 gene, such that it is not turned off but it does not function normally and is associated with a reduction in FMR1 expression. Examples of a Fragile X-associated disorder that can be treated using the vectors, compositions and methods of the present application include Fragile X Syndrome (or FXS) Fragile X-associated primary ovarian insufficiency (or FXPOI) and Fragile X-associated tremor/ataxia syndrome (or FXTAS).

As used herein, the term “effective amount” of a substance is an amount sufficient to produce a desired effect. In some embodiments, an effective amount of a vector is an amount sufficient to infect a sufficient number of target cells of a target tissue of a subject. In some embodiments, a target tissue is neuronal tissue (e.g., brain tissue, such as, cerebral cortex (CCTX), hippocampus (HIPPO), thalamus (TH), inferior colliculus (IC), olfactory bulb (OB), anterior olfactory nucleus (AON), hypothalamus (HT), cerebellum (CBL), etc.). In some embodiments, an effective amount of a vector or composition may be an amount sufficient to have a therapeutic benefit in a subject, e.g., to increase or replace the expression of a gene or protein (e.g., FMRP), to extend the lifespan of a subject, to improve in the subject one or more symptoms of disease (e.g., a symptom of a Fragile X-associated disorder, such as FXS), etc. The effective amount will depend on a variety of factors such as, for example, the species, age, weight, health of the subject, and the mode or site of administration, and may thus vary among subjects and administrations.

As used herein, the terms “protein” and “polypeptide” are used interchangeably and thus the term polypeptide may be used to refer to a full-length protein and may also be used to refer to a fragment of a full-length protein, and/or functional variants thereof. As used herein, the terms “polynucleotide” and “nucleic acid sequence” may be used interchangeably and may comprise genetic material including, but not limited to: RNA, DNA, mRNA, cDNA, etc., which may include full length sequences, functional variants, and/or fragments thereof

The present application provides a therapy for Fragile X-associated disorders. This is based on a gene therapy approach to deliver a transgene to increase or create FMRP function. In particular, the therapy includes methods and compositions for treating a Fragile X-associated disorder, such as FXS, by replacing a missing FMRP in a subject using viral vector-mediated gene therapy.

The genetic construct is a vector based on an adeno-associated virus (AAV) genome that comprises a polynucleotide sequence encoding an FMRP isoform or a variant thereof. This polynucleotide sequence is also referred to herein as the “transgene.” Accordingly, also provided herein are viral vectors useful in the treatment of a Fragile X-associated disorder, such as FXS, and pharmaceutical compositions containing a viral vector useful in the treatment of a Fragile X-associated disorder, such as FXS. The method and compositions of the present application are useful in gene therapy to mitigate some or all of the CNS abnormalities associated with a Fragile X-associated disorder, such as FXS.

Previous studies have shown that AAV-mediated FMR1 gene delivery can be used to reduce the phenotypic severity of FXS in Fmrl knock out mice (Gholizadeh, S. et al. (2014) “Reduced Phenotypic Severity Following Adeno-Associated Virus-Mediated Fmrl Gene Delivery in Fragile X Mice” Neuropsychopharmacology 39: 3100-3111) and to correct abnormally enhanced hippocampal long-term synaptic depression in Fmrl knock out mice (Zeier, Z, et al. (2009) “Fragile X mental retardation protein replacement restores hippocampal synaptic function in a mouse model of fragile X syndrome” Gene Ther 16: 1122-1129). In these previous studies, the FMRP that was expressed by the vectors was mouse isoform 1, which is the full length FMRP. Although expression of FMRP isoform 1 was found to reduce phenotypic severity in Fragile X mice (i.e., Fmrl knock out mice), it was not successful in addressing all, or a majority, of the symptoms associated with FXS as measured using behavioural studies. Furthermore, use of the vector did not result in good expression of the FMRP isoform 1 in all brain regions tested.

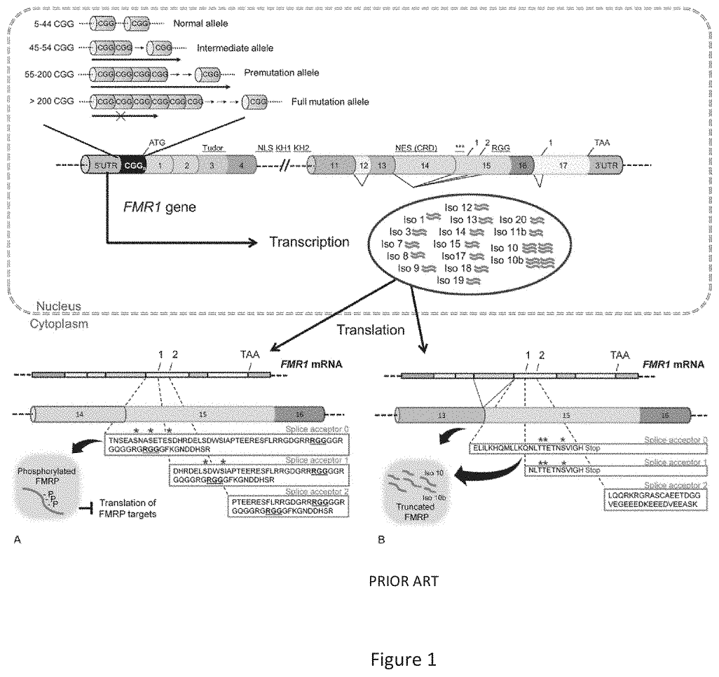

The FMR1 gene produces a variety of structurally related, but functionally diverse protein isoforms as a result of alternative splicing. Studies have demonstrated that the FMR1 sequence and this alternative splicing is highly conserved between species, including between human and mouse (e.g., Ashley, et al. (1993) Nature 4: 244-251). FIG. 1 schematically depicts the alternative splicing pattern of human FMR1 to generate at least 16 human FMRP isoforms (Pretto, D. I., et al. (2015)). These have been generally classified into four groups: Group A isoforms include all of the exons, but have alternative splicing sites within exon 15 and 17; Group B isoforms lack exon 14 and have alternative splicing sites within exon 15 and 17; Group C isoforms lack exon 12 and have alternative splicing sites within exon 15 and 17; and Group D isoforms lack both exon 12 and exon 14, and have alternative splicing sites within exon 15 and 17 (Pretto, D. I., et al. (2015)).

Using mouse and human brain tissues, it has been previously shown that these isoforms, and their mRNA transcripts, exhibit differences with respect to their spatial and temporal expression (Brackett et al. (2013) PloS One 8(3): e58296; and Pretto, D. I., et al. (2015)). Although the specific function of FMRP, and FMRP isoforms, has not been elucidated, the isoform expression pattern suggests that the relative levels of the FMR1 isoforms are modulated according to developmental stage; which is consistent with the severe impact associated with the loss of all protein isoforms found in FXS patients.

Transcripts of Group C isoforms (i.e., those that lack exon 12 and include exon 14; these include isoforms 7, 8, 9, 17, 18, and 19; Pretto, D. I., et al. (2015)) have been found to be expressed more during the later stages of brain development and are the most highly expressed of those transcripts that are expressed in the brain, including the cerebellum. This is in contrast with the full length FMRP isoform 1, which is only minimally expressed in both normal mouse and normal human brain, irrespective of developmental stage.

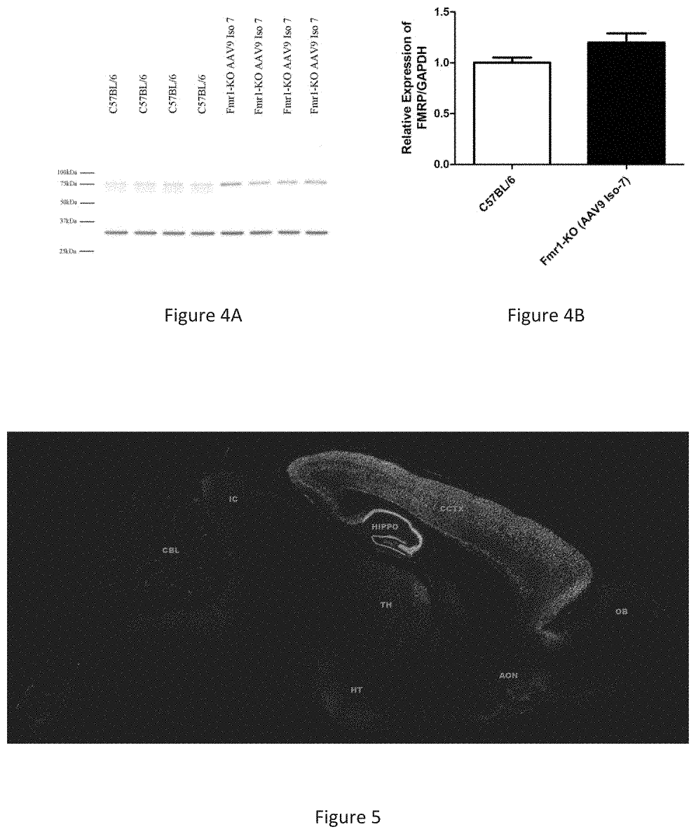

The present inventors sought to improve on the previous studies using vector mediated delivery of FMRP isoform 1 and have surprisingly determined that the use of vector mediated delivery of a gene encoding a Group C FMRP isoform was successful in providing broad expression of the Group C FMRP isoform in the brain tissue of treated subjects. In a specific example, an AAV-FMRP encoding isorform 7 was found to successfully express a protein that co-migrates on SDS-PAGE/western blot with the prominent higher molecular weight form expressed in mouse brain (FIGS. 4A-4B). This also confirmed that FMRP isorform 7 protein is a major form expressed in brain. Expression of a major form of FMRP, for example a Group C isoform (e.g., mouse isorform 7 or human 17), is expected to give superior beneficial therapeutic effects compared to minor forms (e.g., isoform 1).

Recombinant Adeno-Associated Viral (AAV) Vectors

The vector of the present application comprises an adeno-associated virus (AAV) genome or a derivative thereof and a transgene encoding a Group C FMRP isoform, as defined below. The vector is referred to herein as an AAV-FMRP vector.

An AAV genome is a polynucleotide sequence that encodes functions needed for production of an AAV viral particle. These functions include those operating in the replication and packaging cycle for AAV in a host cell, including encapsidation of the AAV genome into an AAV viral particle. Naturally occurring AAV viruses are replication-deficient and rely on the provision of helper functions in trans for completion of a replication and packaging cycle. Accordingly, the AAV genome of the AAV-FMRP vector is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression. One manifestation of double-stranded AAV is through the use of self-complementary AAV (scAAV) vectors.

The AAV genome may be from any naturally derived serotype or isolate or Glade of AAV. Thus, the AAV genome may be the full genome of a naturally occurring AAV virus. As is known to the skilled person, AAV viruses occurring in nature may be classified according to various biological systems.

Commonly, AAV viruses are referred to in terms of their serotype. A serotype corresponds to a variant subspecies of AAV, which, owing to its profile of expression of capsid surface antigens, has a distinctive reactivity that can be used to distinguish it from other variant subspecies. Typically, a virus having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11, also recombinant serotypes, such as Rec2 and Rec3, which have been identified from primate brain.

Examples of serotypes of AAV for use in the AAV-FMRP vector are AAV2 and AAV9, although it should be recognized that other AAV serotypes can also be used. Reviews of AAV serotypes may be found in Choi et al (2005) Curr Gene Ther 5(3); 299-310 and Wu et al (2006) Molecular Therapy 14(3), 316-327.

The skilled person can select an appropriate serotype, Glade, clone or isolate of AAV for use in the present vector on the basis of their common general knowledge. It should be understood, however, that also encompassed herein is the use of an AAV genome of other serotypes that may not yet have been identified or characterised. The AAV serotype can assist in determining the tissue specificity of infection (or tropism) of an AAV virus. Accordingly, preferred AAV serotypes for use in the AAV-FMRP to be administered to patients, as described herein, are those which have natural tropism for or a high efficiency of infection of target cells within the brain and CNS. Depending on the mode of administration, it may also be advantageous for the AAV serotype to have the ability to cross the blood-brain barrier (e.g., AAV9).

Typically, the AAV genome of a naturally derived serotype or isolate or Glade of AAV comprises at least one inverted terminal repeat sequence (ITR). An ITR sequence acts in cis to provide a functional origin of replication and allows for integration and excision of the vector from the genome of a cell. In certain embodiments, one or more ITR sequences flank the polynucleotide sequence encoding the Group C FMRP isoform, or a variant thereof. Preferred ITR sequences are those of AAV2, and variants thereof. The AAV genome typically also comprises packaging genes, such as rep and/or cap genes that encode packaging functions for an AAV viral particle. The rep gene encodes one or more of the proteins Rep78, Rep68, Rep52 and Rep40 or variants thereof The cap gene encodes one or more capsid proteins such as VP1, VP2 and VP3 or variants thereof. These proteins make up the capsid of an AAV viral particle. Capsid variants are discussed below.

As discussed above, the AAV genome used in the AAV-FMRP vector may be the full genome of a naturally occurring AAV virus. For example, a vector comprising a full AAV genome may be used to prepare AAV virus in vitro. However, while such a vector may in principle be administered to patients, this will be done rarely in practice. Preferably the AAV genome will be derivatised for the purpose of administration to patients. Such derivatisation is standard in the art and the present application encompasses the use of any known derivative of an AAV genome, and derivatives which could be generated by applying techniques known in the art. Derivatisation of the AAV genome and of the AAV capsid are reviewed in Coura and Nardi (2007) Virology Journal 4:99, Lisowski, et al. (2015) Curr Opin Pharmacol. 24:59-67 and in Choi et al and Wu et al, referenced above.

Derivatives of an AAV genome include any truncated or modified forms of an AAV genome that allow for expression of a Group C FMRP isoform from a vector in vivo. Typically, it is possible to truncate the AAV genome significantly to include minimal viral sequence yet retain the above function. This is can be beneficial for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or one or more of the ITRs may be a chimeric or mutant ITR. An example of a mutant ITR is one having a deletion of a trs (terminal resolution site). This deletion allows for continued replication of the genome to generate a single-stranded genome which contains both coding and complementary sequences i.e., a self-complementary AAV genome. This allows for bypass of DNA replication in the target cell, and so enables accelerated transgene expression.

The one or more ITRs may flank the polynucleotide sequence encoding a Group C FMRP isoform, or a variant thereof, at either end. The inclusion of one or more ITRs is may be useful to aid concatamer formation of the vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In certain embodiments, ITR elements will be the only sequences retained from the native AAV genome in the derivative. Thus, a derivative may not include the rep and/or cap genes of the native genome and any other sequences of the native genome. This may be beneficial for the reasons described above, and also to reduce the possibility of integration of the vector into the host cell genome. Additionally, reducing the size of the AAV genome allows for increased flexibility in incorporating other sequence elements (such as regulatory elements) within the AAV-FMRP vector in addition to the transgene.

In some embodiments, including in vitro embodiments, AAV derivatives may additionally include one or more rep and/or cap genes or other viral sequences of an AAV genome. Naturally occurring AAV virus integrates with a high frequency at a specific site on human chromosome 19, and shows a negligible frequency of random integration, such that retention of an integrative capacity in the vector may be tolerated in a therapeutic setting.

Where a derivative genome comprises genes encoding capsid proteins i.e. VP1, VP2 and/or VP3, the derivative may be a chimeric, shuffled or capsid-modified derivative of one or more naturally occurring AAV viruses. In particular, the invention encompasses the provision of capsid protein sequences from different serotypes, clades, clones, or isolates of AAV within the same vector, i.e., pseudotyping.

Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV viral vector comprising a naturally occurring AAV genome, such as that of AAV2. Increased efficiency of gene delivery may be affected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

The present application additionally encompasses the provision of sequences of an AAV genome in a different order and configuration to that of a native AAV genome. Other embodiments also encompass the replacement of one or more AAV sequences or genes with sequences from another virus or with chimeric genes composed of sequences from more than one virus. Such chimeric genes may be composed of sequences from two or more related viral proteins of different viral species.

The present AAV-FMRP vector takes the form of a polynucleotide sequence comprising an AAV genome or derivative thereof and a sequence encoding a Group C FMRP isoform, or a variant thereof

For the avoidance of doubt, the invention also provides an AAV viral particle comprising an AAV-FMRP vector. Such AAV particles are referred to as AAV-FMRP viral particles, and can include transcapsidated forms wherein an AAV genome or derivative having an ITR of one serotype is packaged in the capsid of a different serotype. The AAV particles can also include mosaic forms wherein a mixture of unmodified capsid proteins from two or more different serotypes makes up the viral envelope. The AAV particle also includes chemically modified forms bearing ligands adsorbed to the capsid surface. For example, such ligands may include antibodies for targeting a particular cell surface receptor.

Also provided herein is a host cell comprising an AAV-FMRP vector or AAV-FMRP viral particle.

Fragile X Mental Retardation Protein

As indicated above, the AAV-FMRP vector further comprises a polynucleotide sequence encoding a Group C FMRP isoform, or a variant thereof

Examples of human Group C FMRP isoforms include isoforms 7, 17, 8, 18, 9 and 19. Each of these isoforms is missing exon 12. Isoforms 7, 8 and 9 differ from one another by alternative splicing in exon 15. Isoforms 7 and 17 differ from one another by alternative splicing in exon 17. Similarly, isoforms 8 and 18, and 9 and 19, differ from one another, respectively, by alternative splicing in exon 17 Pretto, D. I., et al. (2015)).

Examples of mouse Group C FMRP isoforms include isoforms 7, 8 and 9, which are each missing exon 12 and differ from one another by alternative splicing in exon 15. Sequence alignment shows that mouse isorform 7 corresponds with human isoform 17.

As described above, the specific function of FMRP has not been fully elucidated. However, the impact of a reduction in FMRP expression or a total lack of expression of FMRP is significant abnormalities in brain development, as well as other non-neurological symptoms. Consequently, it is apparent that FMRP expression is critical to proper brain development. The Group C FMRP isoform, or other variant thereof, expressed from the transgene of AAV-FMRP vector functions to replace normal FMRP activity.

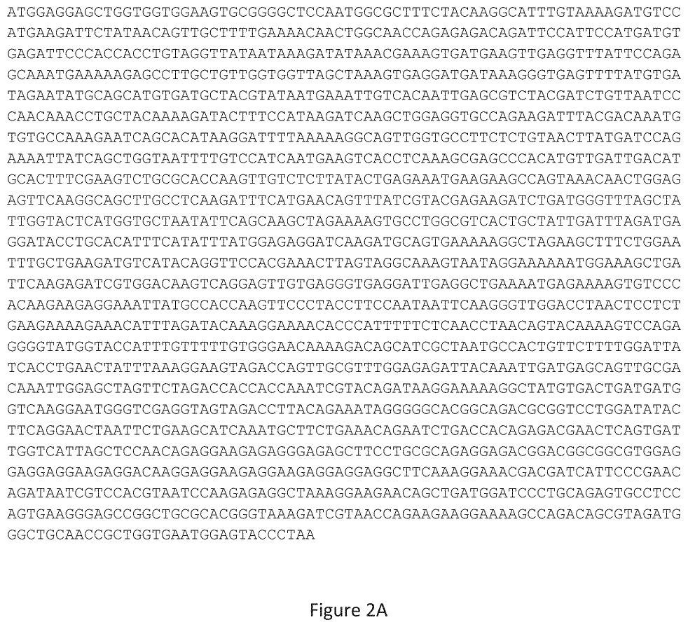

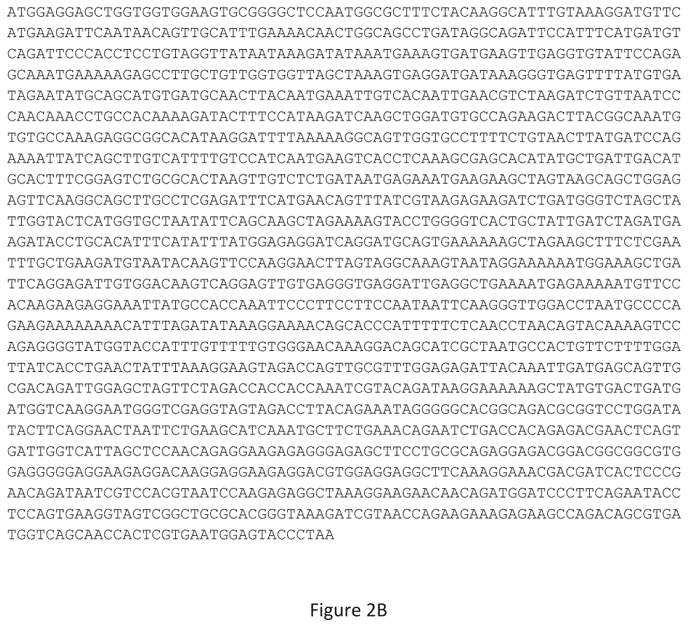

In one embodiment the transgene in the AAV-FMRP vector encodes mouse isoform 7, or an equivalent or similar variant thereof. In one example of this embodiment, the AAV-FMRP transgene comprises the polynucleotide sequence shown in FIG. 2A, or a variant thereof. In another embodiment the transgene in the AAV-FMRP vector encodes human isoform 17. In one example of this embodiment, the AAV-FMRP transgene comprises the polynucleotide sequence shown in FIG. 2B, or a variant thereof

As used herein, a variant polynucleotide sequence is a polynucleotide sequence that enclodes a variant polypeptide that is at least 55%, 65%, 70%, 75%, 80%, 85%, 90% and more preferably at least 95%, 97% or 99% homologous to a relevant region of the naturally occurring sequence over at least 20, preferably at least 30, for instance at least 40, 60, 100, 200, 300, 400 or more contiguous amino acids, or even over the entire sequence of the variant. The relevant region will be one which provides for functional activity of the Group C FMRP isoform.

Homology can be measured using known methods. For example, the UWGCG Package provides the BESTFIT™ program, which can be used to calculate homology (for example used on its default settings) (Devereux et al. (1984) Nucleic Acids Research 12, 387-395). The PILEUP and BLAST algorithms can be used to calculate homology or line up sequences (typically on their default settings), for example as described in Altschul S. F. (1993) J Mol Evol 36:290-300; Altschul, S, F et al. (1990) J Mol Biol 215:403-10. Software for performing BLAST analyses is publicly available through the National Center for Biotechnology Information (http://www.ncbi.nlm.nih.gov/).

A variant Group C FMRP isoform may have a percentage identity with a particular region of the naturally occurring polypeptide sequence, which is the same as any of the specific percentage homology values (i.e., it may have at least 70%, 80% or 90% and more preferably at least 95%, 97% or 99% identity) across any of the lengths of sequence mentioned above.

Variants of Group C FMRP isoform also include truncations. Any truncation may be used so long as the variant is still able to provide Group C FMRP isoform activity. Truncations will typically be made to remove sequences that are non-essential for activity and/or do not affect conformation of the folded protein. Appropriate truncations can routinely be identified by systematic truncation of sequences of varying length from the N- or C-terminus.

Variants of Group C FMRP isoforms further include mutants which have one or more, for example, 2, 3, 4, 5 to 10, 10 to 20, 20 to 40 or more, amino acid insertions, substitutions or deletions with respect to a particular region of of the naturally occurring polypeptide sequence.

Substitutions preferably introduce one or more conservative changes, which replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative change may introduce another amino acid that is aromatic or aliphatic in the place of a pre-existing aromatic or aliphatic amino acid. Conservative amino acid changes are well known in the art and may be selected in accordance with the properties of the 20 main amino acids.

Promoters and Regulatory Sequences

The AAV-FMRP vector also includes elements allowing for the expression of the Group C FMRP isoform transgene in vitro or in vivo. Thus, the vector typically comprises a promoter sequence operably linked to the polynucleotide sequence encoding the Group C FMRP isoform, or variant thereof.

Any suitable promoter may be used. The promoter sequence may be constitutively active i.e., operational in any host cell background, or alternatively may be active only in a specific host cell environment, thus allowing for targeted expression of the transgene in a particular cell type. The promoter may show inducible expression in response to presence of another factor, for example a factor present in a host cell. In any event, where the vector is administered for therapy, the promoter must be functional in the CNS, for example, in the brain.

In some embodiments, it is preferred that the promoter shows CNS specific expression in order to allow for the transgene to only be expressed in CNS cell populations. Thus, expression from the promoter may be CNS-cell specific, such as neuron-specific or neuron-selective expression.

Examples of promoters suitable for use in neuron-specific or selective expression are the synapsin gene promoter (Gholizadeh S., et al. (2014) Neuropsychopharm 39:3100-3111) the methyl CpG binding protein 2 (MeCP2) promoter, and the mini-MeCP2 promoter (Adachi M. et al. (2005) Hum Mol Genet. 14(23): 3709-3722).

The AAV-FMRP vector may also comprise one or more additional regulatory sequences with may act pre- or post-transcriptionally. Regulatory sequences are any sequences that facilitate expression of the transgene (i.e., act to increase expression of a transcript, improve nuclear export of mRNA or enhance its stability). Such regulatory sequences include for example enhancer elements, postregulatory elements and polyadenylation sites. In the context of the AAV-FMRP vector described herein such regulatory sequences will be cis-acting. However, the present application also encompasses the use of trans-acting regulatory sequences located on additional genetic constructs.

An example of a postregulatory element for use in an AAV-FMRP vector is the woodchuck hepatitis postregulatory element (WPRE) or a variant thereof. The invention encompasses the use of any variant sequence of the WPRE that increases expression of the Group C FMRP isoform transgene compared to a vector without a WPRE.

Additional regulatory sequences may be selected by the skilled person on the basis of their common general knowledge.

Preparation of Vector

The AAV-FMRP vector may be prepared by standard means known in the art for provision of vectors for gene therapy. Thus, well established public domain transfection, packaging and purification methods can be used to prepare a suitable vector preparation.

As discussed above, an AAV-FMRP vector may comprise the full genome of a naturally occurring AAV virus in addition to a polynucleotide sequence encoding a Group C FMRP isoform, or a variant thereof. However, commonly a derivatised genome will be used, for instance a derivative that has at least one inverted terminal repeat sequence (ITR), but which may lack any AAV genes such as rep.

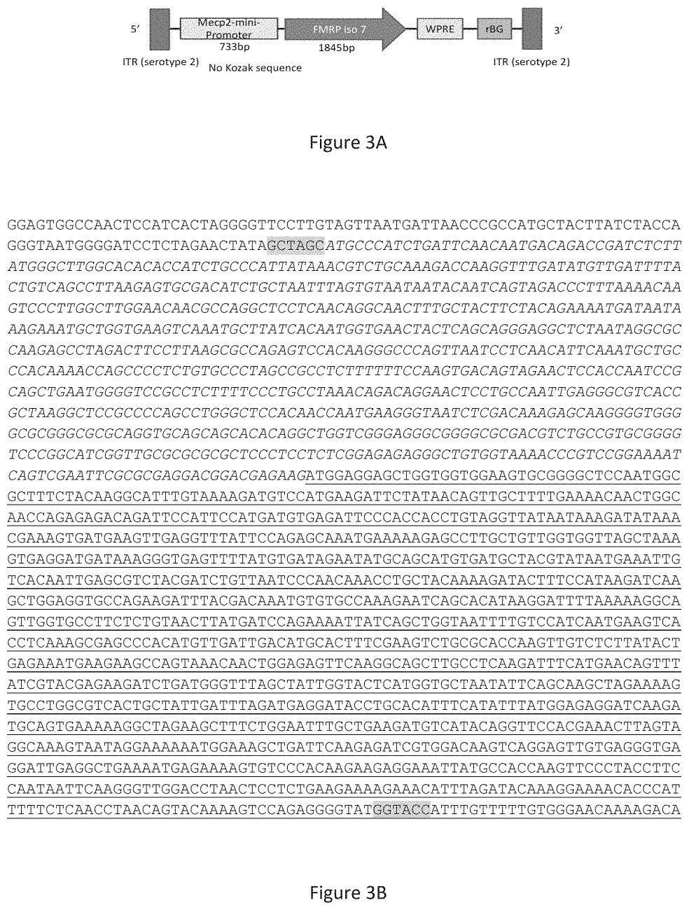

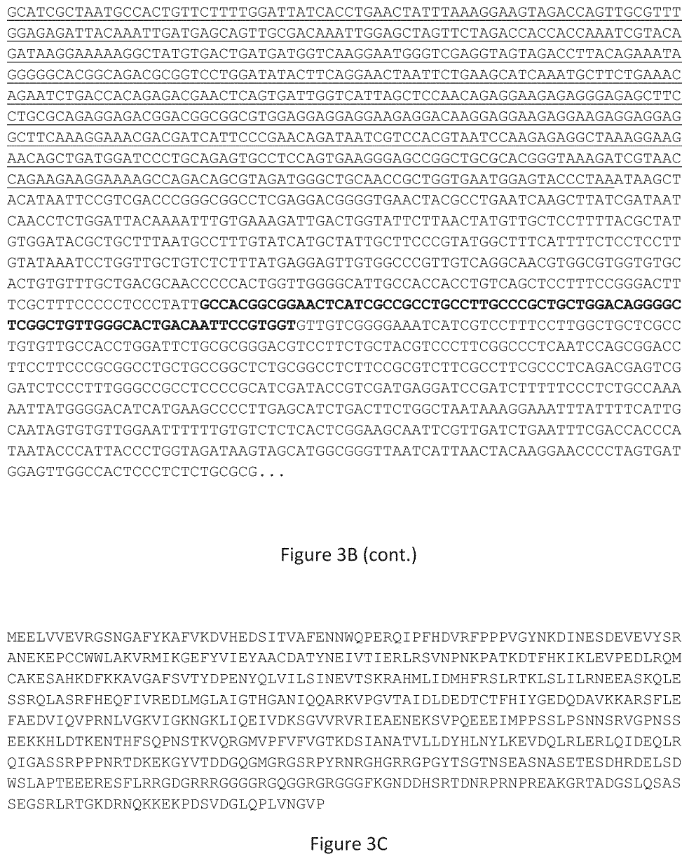

One example of an AAV-FMRP vector comprises a derivatised genome of AAV2 in combination with AAV9capsid proteins. This packaged viral vector typically comprises one or more AAV2 ITRs optionally as shown in FIGS. 3A and 3B.

In these aspects, the invention provides a method for production of an AAV-FMRP vector. The method comprises providing a vector which comprises an adeno-associated virus (AAV) genome or a derivative thereof and a polynucleotide sequence encoding a Group C FMRP isoform or a variant thereof in a host cell, and providing means for replication and assembly of said vector into an AAV viral particle. Typically, the derivative of an AAV genome comprises at least one ITR, commonly derived from the AAV2 serotype. Optionally, the method further comprises a step of purifying the assembled viral particles. Additionally, the method may comprise a step of formulating the viral particles for therapeutic use.

Methods of Therapy and Medical Uses

As discussed above, the present inventors have surprisingly demonstrated that an AAV-FMRP vector may be used to increase or generate Group C FMRP isoform expression. This provides a means whereby the reduction or absence of FMRP expression associated with Fragile X-associated disorders, such as FXS, can be replaced with Group C FMRP isoform expression in order to treat, arrest, or prevent the symptoms associated with Fragile X-associated disorders, such as FXS.

The invention therefore provides a method of treating or preventing a Fragile X-associated disorder, such as FXS, in a subject in need thereof, comprising administering a therapeutically effective amount of an AAV-FMRP vector to the subject by, for example, direct injection into the cerebral spinal fluid via intra-cerebroventricular injection or intra-cisterna magna injection, and/or intrathecal injections. Accordingly, a Fragile X-associated disorder, such as FXS, can be treated, arrested or prevented in said subject.

In a related aspect, the invention provides for use of an AAV-FMRP vector for treating or preventing a Fragile X-associated disorder, such as FXS by administering said vector to a patient by, for example, direct intra-cerebroventricular injection or intra-cisterna magna injection. Additionally, the invention provides the use of an AAV-FMRP vector in the manufacture of a medicament for treating or preventing a Fragile X-associated disorder, such as FXS by administering said vector to a patient by, for example, direct intra-cerebroventricular injection or intra-cisterna magna injection, and/or intrathecal injection.

The dose of an AAV-FMRP vector may be determined according to various parameters, especially according to the age, weight and condition of the patient to be treated; the route of administration; and the required regimen. Again, a physician will be able to determine the required route of administration and dosage for any particular patient.

Pharmaceutical Compositions

The AAV-FMRP vector can be formulated into a pharmaceutical composition. Such a composition may comprise, in addition to the vector, a pharmaceutically acceptable excipient, carrier, buffer, stabiliser or other materials well known to those skilled in the art. Such materials should be non-toxic and should not interfere with the efficacy of the active ingredient (i.e., the AAV-FMRP vector). The precise nature of the carrier or other material may be determined by the skilled person according to the route of administration (e.g., here direct intra-cerebroventricular injection or intra-cisterna magna injections).

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used.

For injection, the active ingredient will be in the form of an aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

For delayed release, the vector may be included in a pharmaceutical composition which is formulated for slow release, such as in microcapsules formed from biocompatible polymers or in liposomal carrier systems according to methods known in the art.

To gain a better understanding of the invention described herein, the following examples are set forth. It should be understood that these examples are for illustrative purposes only. Therefore, they should not limit the scope of this invention in any way.

### EXAMPLES

Materials and Methods

All studies were carried out using C57/BL6J wild-type mice and Fmrl KO mice backcrossed 10 generations on C57/BL6J mice. All procedures were approved by the University of Toronto Animal Care Committee and were carried out in compliance with the Canadian Council on Animal Care guidelines.

The studies provided below employed an example of an AAV-FMRP vector that is a single-stranded AAV vector containing the inverted terminal repeat (ITR) DNA sequences from the genome of AAV2 serotype, the capsid protein genes from AAV9serotype, and the human Mecp2-mini-promoter. This vector may or may not contain a Kozak sequence and/or the WPRE (woodchuck hepatitis virus post-transcriptional regulatory element) and a recombinant bovine growth hormone (rBG) element. A schematic of this AAV-FMRP vector is provided in FIG. 3A. The partial DNA sequence of this AAV-FMRP vector is shown in FIG. 3B, where the ellipses are used to indicate the remaining sequence of the AAV2 serotype inverted terminal repeats (ITR). Also shown in FIG. 3B is the position of the MeCP2-mini-promoter (italics), the FMRP mouse isorform 7 coding sequence (underlined) and the WPRE (bold). The two small shaded sequences are the restricition enzyme cut sites employed in constructing the vector (the first is an Nhel cut site and the second is a Kpnl cut site). This vector is referred to below as “AAV9-Fmrl-Iso7”.

FIG. 3C depicts the amino acid sequence of the FMRP mouse isorform 7 expressed by AAV9-Fmrl-Iso7.

The AAV9-Fmrl-Iso7 was purified and used at a concentration of 1-9×10¹³ vector genomes/mL in sterile phosphate-buffered saline (PBS) and stored at −80° C.

Vector Injections

Intracerebroventricular and intra cisterna magna injection protocol
Prepare a desired titer of viral vector (2 μL total).
Neonatal Fmrl-KO mice at postnatal day (PND) 2 or 3 are anaesthetized by brief cooling on ice pad.
Injections are performed using a microinjection pump (CMA/100, Carnegie Medicine, Sweden) to operate a 10 μl Hamilton syringe with a 30 G needle with beveled tip.
Place the immobilized mouse on a transilluminator to illuminate relevant anatomical structures that can be used as a guide.
A local anaesthetic is used prior to ICV, ICM, delivery (Marcaine-1 mg/kg, by infiltration). For ICV, the needle is inserted 1 mm deep, perpendicular to the skull surface, at a location approximately 0.25 mm lateral to the sagittal suture and 0.50-0.75 mm rostral to the neonatal coronary suture. The needle tip is inserted through the skull using gentle pressure to penetrate the partially formed bony layer and inject 1-3 μl of AAV vector into the brain ventricles at a flow rate of 1 μl/min. For ICM, the needle is inserted according to a brain atlas, 1 mm deep at the base of the skull, angled towards the nose of the animal and aligned with the midline of the brain. A total of 3-10 μl is injected at a rate of 1 μl/min.
The needle is removed slowly after 60 sec after discontinuation of plunger movement to prevent backflow. Mice are allowed to recover for 5-10 min in a warmed container until movement and general responsiveness is restored.

### Example 1

### Hippocampal Expression Levels of Total FMRP in Treated and Untreated Mice

In this study, hippocampal expression levels of total FMRP were demonstrated to be similar in untreated wild-type mice and in Fmrl-KO mice injected with AAV9-Fmrl-Iso7.

Samples of the mouse hippocampus were dissected from untreated C57BL/6 mice, and Fmrl-KO mice after intra-cerebroventricular and intra-cisterna magna injection with AAV9-Fmrl-Iso7 at PND 2, as described above. Hippocampi were collected at PND 25-32 and prepared for SDS-PAGE electrophoresis using 1 mM dithiotherital, and the proteins were transferred to nitrocellulose for western blotting; the blots were stained with anti-FMRP antibody (5C2, Biolegend, #MMS-5232).

A representative western blot of brain hippocampal lysates (10 μg total protein) from untreated C57BL/6 mice, and Fmrl-KO mice after intra-cerebroventricular and intra-cisterna magna injection with AAV9-Iso7 at P2 is provided in FIG. 4A. Hippocampi were collected at P25-32, and blots were stained with anti-FMRP antibody (5C2, Biolegend, #MMS-5232). In greater detail, after SDS-polyacrylamide gel electrophoresis, the proteins were transferred onto nitrocellulose membranes, blocked with a 5% milk solution in PBS, and incubated overnight with the primary antibody anti-FMRP antibody (5C2, Biolegend, #MMS-5232) 1:1000 dilution with the addition of mouse monoclonal anti-GAPDH (GAPDH-71.1; Sigma-Aldrich) at 1:8,000 dilution which was used as a loading control. Following 3 washes with PBS, the membranes were incubated with HRP-conjugated goat anti-mouse or anti-rabbit secondary antibodies, and SuperSignal West Pico Chemiluminescent Substrate was added to the membranes to reveal the bands. Images were exposed under an Alpha Innotech Fluorochem gel imager.

The Image J software was used to quantify the density of the protein bands. Sample values were averaged from of two quantifications. N value represents the number of animals used. Quantification of protein expression was normalized using GAPDH immunoreactivity and data is presented as a percentage of wild-type control expression. Results are presented as mean±SEM. Statistical significance was determined with Graphpad Prism 5 by using one-way ANOVA and Tukey's test.

The quantification results are shown in FIG. 4B. Quantification of total FMRP (50-75 kDa) showed no significant difference (p>0.05) between the AAV-FMRP treated and untreated wild-type groups. N values: C57BL/6 wild-type=4; Fmrl-KO AAV9-Fmrl-Iso7=4. This study demonstrated that under these conditions levels of injected AAV-FMRP transgene injected in the Fmrl KO mouse brain can approximate levels the FMRP level found naturally in normal unaffected (wild-type) mouse brain.

### Example 2

### Representative Expression of FMRP Isorform 7 Transgene in a Neonatal Fmrl-KO Mouse Pup

Expression of FMRP isorform 7 transgene in a neonatal Fmrl-KO mouse pup was studied one month after i.c.v. plus i.c.m injection of the AAV9-Iso7 into the neonatal Fmrl-KO mouse pup (at PND 2). A sagittal brain section was obtained from the mouse pup at postnatal day (PND) 31. The sagittal brain section was stained with anti-FMRP antibody and visualized via immunofluorescence at PND 31, as described in more detail below.

About 30 days post-injection, the AAV9-Fmrl-Iso7-treated-mice were anesthetized using a Ketamine-Xylazine solution then transcardially perfused with PBS, followed by 4% paraformaldehyde in PBS (pH 7.4) followed by 30% sucrose in PBS for 24-48 hours. Serial coronal sections were cut at a thickness of 25 μm using a cryostat (Leica Microsystems, Wetzlar, Germany). Free-floating sections were washed with PBS and antigen retrieval. Monoclonal mouse anti-FMRP 5c2 was used at a 1: 500 dilution. Prior to experimentations, three different antibody dilutions were used to determine proper antibody titer and to avoid signal saturation (at 1:500, 1:1000, and 1:5000). After overnight incubation, sections were washed five times with PBS and then secondary antibodies diluted in PBS containing 5% goat serum were applied. The sections were labeled with goat anti-mouse Alexa Fluor 594 (1: 1000; Jackson Immunoresearch). DAPI (4′,6-diamidino-2-phenylindole) was used to label cell nuclei. The images were captured using a laser scanning confocal microscope (Zeiss 700, Carl Zeiss Canada, Toronto, Canada) at 20× objective magnification and analyzed using the Zen 2010 Blue software (Carl Zeiss).

The results show that expression was high in the cerebral cortex (CCTX) and hippocampus (HIPPO), with lower expression in the thalamus (TH), inferior colliculus (IC), olfactory bulb (OB), anterior olfactory nucleus (AON), hypothalamus (HT), and cerebellum (CBL). A representative photomicrograph, showing this expression pattern, is provided in FIG. 5.

### Example 3

### Natural Cellular Co-Distribution of MeCP2 and FMRP Protein Expression in Wild-Type Mouse Brain

This study was performed to demonstrate the natural co-distribution of MeCP2 and FMRP expression in wild-type mouse brain. A sagittal brain section was obtained from a C57/BL6 wild-type mouse at PND 31, using the same technique as described in Example 2. The sagittal brain section was stained with anti-FMRP antibody and anti-MeCP2 antibody (rabbit monoclonal from Cell Signaling Technology, 1 to 1000 dilution) and visualized via immunofluorescence. After overnight incubation, five washes with TBS for 10 min each were carried out and secondary antibodies diluted in TBS containing 5% goat serum were applied. The sections incubated with anti-FMRP were labeled with goat anti-mouse Alexa Fluor 488 and goat anti-rabbit Alexa Fluor 594 (1:1000; Jackson ImmunoResearch Laboratories. West Grove, PA). The images were captured using a laser-scanning confocal microscope at 10, 40, or ×100 magnifications and analyzed using the NIS-Elements software (Nikon). The images for sagittal sections were captured using a Zeiss Mirax slide scanner at ×20 magnification.

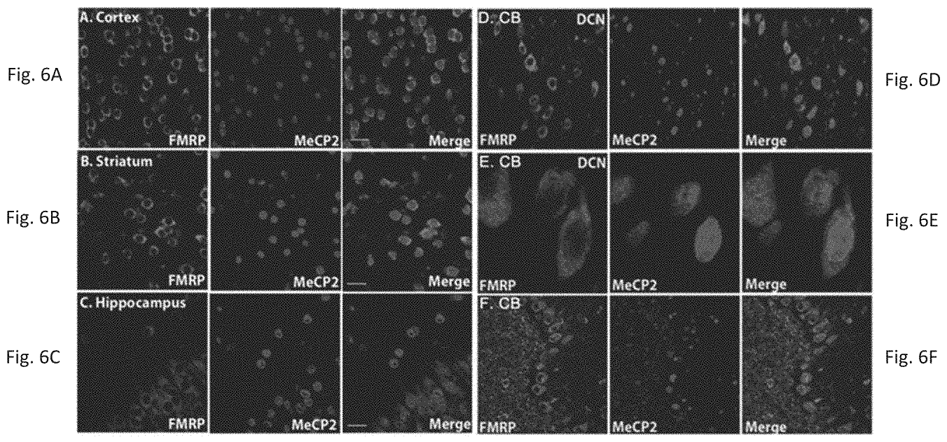

Anti-FMRP labeling was done in green and anti-MeCP2 was done in red (shown in grey scale in FIG. 6A-6D). Both proteins are typically expressed within the same cells. FIGS. 6A-6D provide photomicrographs of thin sections from the cerebral cortex (FIG. 6A), striatum (FIG. 6B), and hippocampus (FIG. 6C). FIGS. 6D and 6E are photomicrographs of the deep cerebellar nuclei (DCN) under low (D) and high magnification (FIG. 6E). FIG. 6F shows an example of cerebellar cortex showing the Purkinje cell layer situated between the molecular layer (on the right side in each panel of FIG. 6F) and granule cell layer (left side of each panel in FIG. 6F). Note that in panels 6A-6F MeCP2 is localized mainly to neuronal nuclei while FMRP is localized mainly to the cytosol compartment within the same neurons.

Overall results demonstrate that the promoter mini-MeCP2 is appropriate for directing expression of the FMRP, from a vector, within the brain tissue in a pattern that is consistent with wild-type expression of FMRP.

### Example 4

### Immunocytochemical Analysis of AAV-FMRP Expression

In this study, immunocytochemical analysis of FMRP expression from AAV9-Iso7 was performed in thin sections of brain using anti-FMRP with anti-cell type marker antibodies and double-label immunofluorescence.

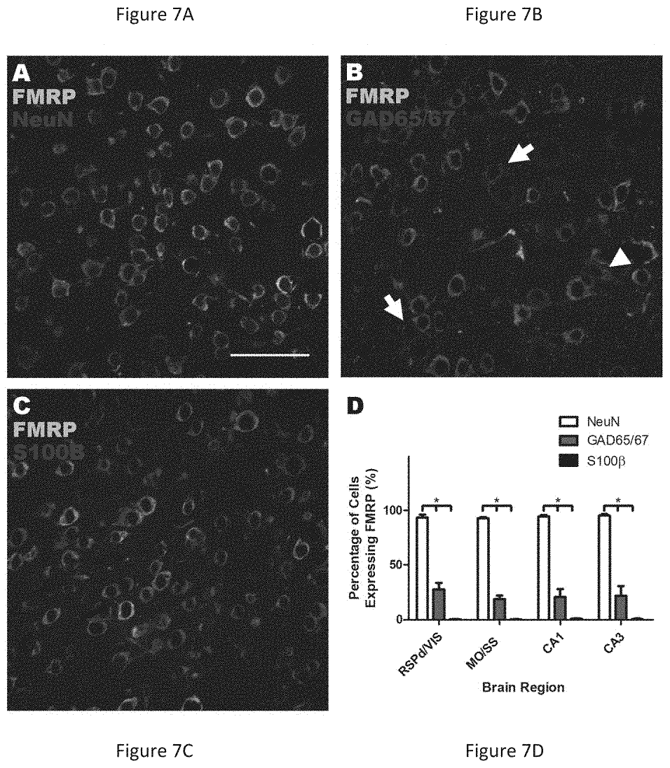

Neonatal Fmrl KO mice were treated with AAV9-Fmrl-Iso7, via i.c.v. injection, as described above. One month later the brains were prepared for immunolabeling, as described in Example 2. Results of immunocytochemical analysis are provided in FIGS. 7A-7D. FIGS. 7A-C are representative images of cerebral cortex labelled for NeuN (all neurons), GAD65/67 (GABAergic neurons only), and S100β (glia), respectively, and co-labelled for FMRP (5C2). The two arrows in FIG. 7B indicate examples of cells positively stained for GAD65/67 and the arrowhead indicates an example of a GAD65/67 positive cell co-labelled for FMRP. FIG. 7D shows the quantification of FMRP and cell type marker co-localization by brain region (RSP/VIS=retrosplenial/visual cortex, SS/MO=somatosensory/motor cortex, CA1 =hippocampus CA1 region, CA3 =hippocampus CA3 region). These results are presented as averages±SEM (shown with error bars) and statistical significance was determined using one way ANOVA and Tukey's HSD (Tukey's “Honest Significant Difference” method).

Transduced FMRP was found to be highly expressed in NeuN positive cells, moderately expressed in GAD65/67 positive cells, and was almost completely undetectable in S100β positive cells. These results demonstrate that the AAV-FMRP transgene expression mimicked the natural pattern of FMRP distribution in the adult brain (i.e., highly expressed in neurons with little expression in glial cells).

### Example 5

### FMRP Expression in Liver Following Transduction

In this study, neonatal Fmrl-KO mice received i.c.v. and i.c.m. injections with either the AAV-FMRP vector (mini-MeCP2-Fmrl-Iso7-AAV9) or an AAV9-CMV-EV (where CMV is a cytomegalovirus promoter) control vector at PND 2. The AAV9-CMV-EV is an empty vector containing no transgene, which was used as the appropriate negative control.

At PND 25-PND 32 the AAV-FMRP treated mice and wild-type mice were anesthetized using a Ketamine-Xylazine solution then transcardially perfused with PBS, followed by 4% paraformaldehyde in PBS (pH 7.4). Livers were collected and liver lysates were prepared using SDS-PAGE sample buffer; 10 μg total protein were used in western blots stained with anti-FMRP antibody (5C2, from Biolegend Inc.).

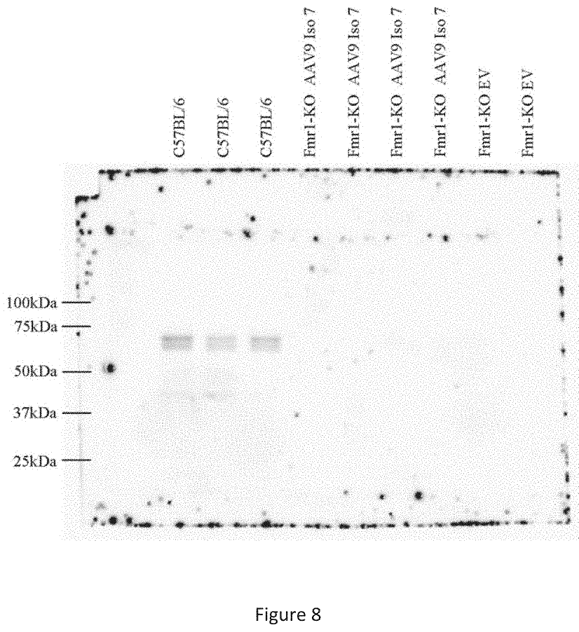

This study found that FMRP is not expressed in the liver after transduction with AAV9-Iso7. A representative western blot of liver lysates is provided in FIG. 8. FMRP isoform bands were visible between 50-75 kDa in C57 wild-type mice (3 lanes on left), compared to Fmrl KO mice injected with AAV9-Iso7 or AAV-EV where no FMRP transgene was detected. This study demonstrates that AAV9-Fmrl-Iso7 injected into the cerebral spinal fluid of the mouse does not induce FMRP transgene expression in the liver.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A vector comprising an adeno-associated virus (AAV) genome or a derivative thereof and a nucleic acid sequence encoding a Fragile X Mental Retardation Protein (FMRP) isoform that lacks exon 12 and includes exon 14 of a full length FMRP1 gene.

2. The vector of claim 1, wherein the FMRP isoform is a human or a murine Group C FMRP isoform.

3. The vector of claim 1, wherein the FMRP isoform is human FMRP isoform 17.

4. The vector of claim 1, wherein the FMRP isoform is murine FMRP isorform 7.

5. The vector of claim 1, wherein the nucleic acid encoding the FMRP is operably linked to a neuron-selective promoter.

6. The vector of claim 5, wherein the neuron-selective promoter is a MECP2 promoter, a mini-MECP2 promoter, or a human synapsin mini-promoter.

7. The vector of claim 1, wherein the vector comprises a derivative of an AAV genome.

8. The vector of claim 7, wherein the derivative of the AAV genome comprises at least one AAV serotype 2 ITR region and an AAV serotype 9 capsid sequence.

9. The vector of claim 1, which comprises one or more additional regulatory sequences.

10. The vector of claim 1 for use in treating or preventing a Fragile X-associated disorder in a subject in need thereof.

11. A viral particle comprising the vector of claim 1.

12. The viral particle of claim 11, wherein the nucleic acid encoding the FMRP is operably linked to a neuron-selective promoter that is a MECP2 promoter, a mini-MECP2 promoter, or a human synapsin mini-promoter.

13. A host cell comprising the vector of claim 1.

14. The host cell of claim 13, wherein the nucleic acid encoding the FMRP is operably linked to a neuron-selective promoter that is a MECP2 promoter, a mini-MECP2 promoter, or a human synapsin mini-promoter.

15-16. (canceled)

17. A pharmaceutical composition of claim 1 and a pharmaceutically acceptable carrier or excipient.

18. The pharmaceutical composition of claim 17, wherein the composition is formulated for intra-cerebroventricular injection, intra-cisterna magna injection, and/or intrathecal injection.

19. A method of treating a Fragile X-associated disorder in a patient comprising administering the vector according to claim 1 to the patient.

20. The method according to claim 19, wherein the vector is administered to the patient by intra-cerebroventricular injection, intra-cisterna magna injection, and/or intrathecal injection.

21. The method according to claim 19, wherein the nucleic acid encoding the FMRP is operably linked to a neuron-selective promoter that is a MECP2 promoter, a mini-MECP2 promoter, or a human synapsin mini-promoter.

22. The pharmaceutical composition according to claim 17, wherein the nucleic acid encoding the FMRP is operably linked to a neuron-selective promoter that is a MECP2 promoter, a mini-MECP2 promoter, or a human synapsin mini-promoter.
